(19) United States
(12) Patent Application Publication
(10) Pub. No.: US 2024/0090896 A1
(43) Pub. Date: Mar. 21, 2024

(54) ENDOSCOPIC SUTURING DEVICE AND ENDOSCOPIC SUCTION AND SUTURING CHAMBER

(71) Applicant: Sociedade Beneficente de Senhoras Hospital Sírio Libanês, São Paulo - SP (BR)

(72) Inventor: Kiyoshi Hashiba, São Paulo - SP (BR)

(21) Appl. No.: 18/468,372

(22) Filed: Sep. 15, 2023

(30) Foreign Application Priority Data
Sep. 16, 2022 (BR) 102022018648-0

(51) Int. Cl. A61B 17/06

(52) U.S. Cl. CPC A61B 17/06004; A61B 2017/0496

(57) ABSTRACT

The present invention refers to an endoscopic suturing device and to an endoscopic suction and suturing chamber for joining tissues which offers effective and permanent result in therapeutic processes, in addition to the reduction in morbidity and in the costs involved in the surgical procedures. The endoscopic suturing device (10) comprises an elongated tubular element (20) which extends from a tensioning element (30) associable with an elongated tensionable suturing element (40) comprising proximal end (41) and distal end (42), the proximal end (41) comprising suturing elements (50, 51) so that the distal end (42) enters the receptacle (21) for fastening to the tension point (31), and the suturing elements (50, 51) are delimited by the limiting region (22) and rigidly joined to each other when the tensioning element (30) is activated.

### CROSS REFERENCE TO RELATED APPLICATIONS

The present application claims priority to and the benefit of Brazilian Patent Application Serial No. 102022018648-0, filed Sep. 16, 2022; the entire contents of which as are hereby incorporated by reference herein.

### TECHNICAL FIELD

The present invention refers to an endoscopic suturing device and endoscopic suction and suturing chamber for suturing tissues, which shows effective and permanent therapeutic results, apart from the reduction in morbidity and in the costs involved in surgical procedures.

### DESCRIPTION OF THE STATE OF THE ART

Endoscopes are tools which allow examining and/or treating directly, for example, lesions, by means of the insertion of a tubular instrument in the organ to be examined. Endoscopes can be of the most diverse types, for example, endoscopes containing lens and/or camera systems, tubes which allow viewing the organ, etc. The endoscopes present great variations as regards length, caliber, lateral or distal view, with one or two work channels and also filter systems for better identification of the lesions, etc.

Apart from being extremely effective for enabling examinations and diagnosis of diseases, endoscopes are being increasingly used for endoscopic surgeries. Since the end of the last century, several surgical tools have been developed for performing endoscopic surgeries in an organ interior, by means of fitting a suturing equipment at the end of the endoscopic tube.

As a result, the endoscopic surgeries are constantly evolving with the purpose of enabling therapeutic processes inside the esophagus, stomach, duodenum, small and large intestine, and pancreas and bile ducts aiming at reducing, firstly and more importantly, the morbidity and mortality, in said less invasive procedures and, secondly, the cost of treatments. Equipment and procedures reflecting this evolution are found in documents U.S. Pat. Nos. 8,540,735 and 9,788,831.

Notwithstanding, since the use of the endoscopic method was no longer restricted only to the examination of the digestive tract, the therapeutic part surpassed the diagnosis part, wherein the suturing of tissues became a purpose to be achieved, not only for repair of perforations, but mainly for therapeutic processes, for example, the management of gastroesophageal reflux disease (GERD) and of obesity, nowadays known as endoscopic sleeve gastroplasty.

In this sense, as an alternative method to the gastric resection, medicine is currently using endoscopic suturing tools to reduce the intragastric volume by means of endoscopic plications, that is, the plication carried out on the front and rear walls aiming at reducing the stomach's volume, similar to the vertical gastrectomy, causing retraction, however, without the need to remove a part of the organ.

The endoscopic suturing is performed so that the walls are joined by stitches, reducing the width of the organ cavity. A needle traverses the wall at one place or in several points so that the lumen is reduced at the end of the endoscopic procedure, wherein part of the stomach wall is folded, that is plicated, whereby a needle is run through the apprehended parts, joining the parts to reduce the gastric lumen and, consequently, the volume capacity thereof.

The state of the art which reflects this advance can be found in documents U.S. Pat. Nos. 5,887,594 and 9,107,654.

However, said documents describe tools and processes which do no not present satisfactory results, in the sense that, currently, the majority of the plication sutures performed by means of endoscopic plications do not present sufficient approximation under pressure and/or become undone in a short while, so they do not offer a permanent treatment.

Besides the result not being permanent, there exists the possibility of the occurrence of internal lesions in the organ, which, in extreme cases, can lead to the patient's death.

### OBJECTS OF THE INVENTION

In view of the problems described in the state of the art, the present invention provides an endoscopic suturing device which consists of a pressure joining mechanism so that the approximation under pressure causes the sutured tissues to merge after a short while.

The present invention further provides an endoscopic suction and suturing chamber consisting of a pressure joining mechanism so that the pressure approximation causes the sutured tissues to merge after a short while.

Besides the permanent joining, the endoscopic suturing device of the present invention has the purpose of simplifying the execution of the surgical procedure, reducing the chances of occurrence of intragastric and extragastric lesions and, consequently, the morbidity rates among patients, as well as reducing the costs of this procedure.

### BRIEF DESCRIPTION OF THE INVENTION

The present invention provides an endoscopic suturing device comprising an elongated tubular element which extends as from a tensioning element associated to an elongated tensioning suturing element, wherein the elongated tubular element comprises an inlet end provided with a limiting region, the tensioning element comprising a tension point and the elongated tensionable suturing element comprising a tension point and the elongated tensionable suturing element comprising a proximal end and a distal end, the proximal end comprising suturing elements so that the distal end enters the receptacle for fastening to the tension point and the suturing elements are delimited by limiting regions and rigidly joined to each other when the tensioning element is activated.

In an alternative embodiment of the endoscopic suturing device, the suturing elements comprise a passage for at least one suture thread.

In an alternative embodiment of the endoscopic suturing device, the suturing elements comprise a first suturing element rigidly fixed on the proximal end and a second suturing element with free movement around and longitudinally to the elongated tensionable suturing element.

In an alternative embodiment of the endoscopic suturing device, the rigid union between the suturing elements is configured by the detachment of a first suturing element rigidly fixed on the proximal end for mechanical interference fit to a second suturing element with longitudinal movement limited by the limiting region when the tensioning element is activated.

In an alternative embodiment of the endoscopic suturing device, the rigid union between the suturing elements is configured by the detachment of a first suturing element comprising a lower portion provided with a surrounding projection.

In an alternative embodiment of the endoscopic suturing device, the limiting region comprises a stopper extending transversely and toward the elongated tensionable suturing element so as to define a passage for movement of the elongated tensionable suturing element jointly with the limitation of movement of the suturing elements.

In an alternative embodiment of the endoscopic suturing device, the receptacle comprises an upper opening, longitudinal to the elongated tensionable suturing element, so as to configure the movement of at least one suture thread.

In an alternative embodiment of the endoscopic suturing device, the tensioning element comprises an upper opening longitudinal to the elongated tensionable suturing element for fastening of the distal end at the tension point.

In an alternative embodiment of the endoscopic suturing device, the tension point comprises a split body with the lower part rigidly fixed on the tensioning element and the upper part operationally able to be fitted on the lower part.

In an alternative embodiment of the endoscopic suturing device, the tension point comprises a split body with the lower part provided with a groove for coupling with the distal end.

The present invention further provides an endoscopic suturing and suction chamber comprising a tubular body defining an annular side wall and a closing wall, the annular side wall defining an internal space comprising an upper opening which gives access to the internal space, wherein the closing wall is an inclined closing wall with a lower portion which extends in an inclined manner as from the lower end of the annular side wall to an upper portion and meeting with an upper end of the annular side wall.

In an alternative embodiment, the endoscopic suturing and suction chamber comprises the lower portion of the inclined closing wall extending between 20° and 80° as from the lower end of the annular side wall to the upper portion of the inclined closing wall.

### BRIEF DESCRIPTION OF THE DRAWINGS

The present invention will now be described in detail based on the execution examples represented in the drawings. The figures show:

FIG. 1—a first upper view of the endoscopic suturing device of the present invention;

FIG. 2—a second perspective view of the device shown in FIG. 1;

FIG. 3—a third perspective view of the device shown in FIG. 1;

FIG. 4—a first upper view of an application of the device shown in FIG. 1;

FIG. 5—a first side view of a suturing chamber of the state of the art;

FIG. 6—a first perspective view of the endoscopic suturing and suction chamber of the present invention;

FIG. 7—a second perspective view of the chamber shown in FIG. 6;

FIG. 8—a second perspective view of the chamber shown in FIG. 6;

FIG. 9—a first perspective view of an embodiment of the chamber shown in FIG. 6;

FIG. 10—a second perspective view of an embodiment of the chamber shown in FIG. 8.

### DETAILED DESCRIPTION OF THE DRAWINGS

Firstly, it must be noted that the expression “preferred” used in this document must not be understood as “imperative” or “mandatory”. The expression “preferred” is meant to characterize a particularly efficient embodiment of the invention among the multiple possible ones, whereby the scope of the present invention is limited only by the characteristics that are absolutely essential to obtain the proposed technical effects.

For purposes of understanding the present invention, the expressions “elements”, “means” and “components” must be understood as any product which develops the function for which a specific result is expected.

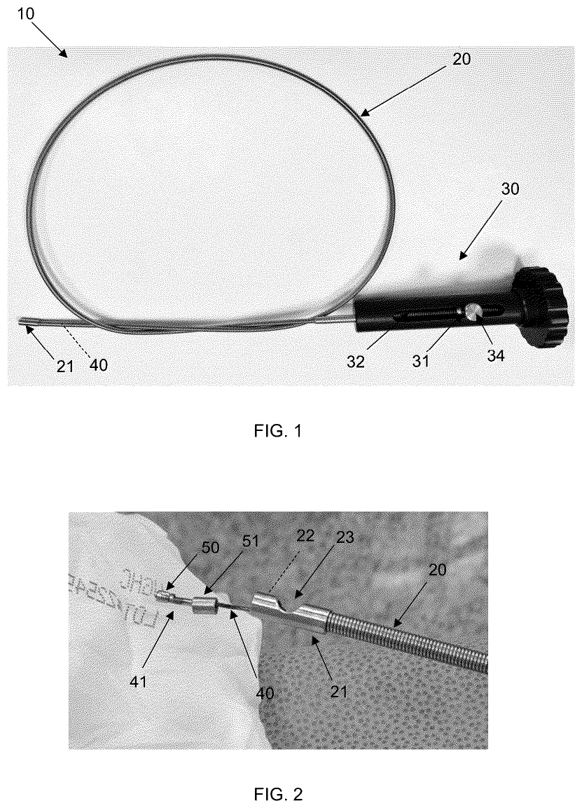

FIGS. 1 to 4 illustrate an embodiment of the endoscopic suturing device of the present invention.

As observed, the endoscopic suturing device 10 comprises an elongated tubular element 20 which extends as from a tensioning element 30, both elements 20, 30 being associable to an elongated tensionable suturing element 40 by means of the insertion of said element 40 in a receptacle 21 at the inlet end of the elongated tubular element 20 to a tension point 31 on tensioning element 30.

The elongated tensionable suturing element 40 comprises a distal end 42 which enters receptacle 21, whereby it is conducted by elongated tubular element 30 until meeting with tension point 31 on tensioning element 30. Naturally, the elongated tensionable suturing element 40 comprises a smaller thickness than the internal area of elongated tubular element 30 and tensioning element 30.

The elongated tensionable suturing element 40 comprises a proximal end 41 associable with suturing elements 50, 51 and which meets with a limiting region 22 in receptacle 21, so that the suturing elements 50, 51 are delimited by the limiting region 22 and rigidly joined to each other when tensioning element 30 is activated.

It must be observed that suturing elements 50, 51 configure a passage for at least one suturing thread ST.

After the association of distal end 42 of the elongated tensionable suturing element 40 on tension point 31 of tensioning element 30, as well as at least one suturing thread ST between suturing elements 50, 51, activating tensioning element 30 results in tensioning of elongated tensionable suturing element 40 so the length of elongated tensionable suturing element 40 corresponds to the length of elongated tubular element 20 plus the distance until the position of tension point 31 on tensioning element 30.

As such, suturing elements 50, 51 and suture thread ST form a set with restricted movement and positioned on the limiting region 22 of receptacle 21, so that, as the activation of tensioning element 30 continues, the tension applied on elongated tensionable suturing element 40 suffices to rigidly join suturing elements 50, 51 and suture thread ST, detaching suturing elements 50, 51 from the endoscopic suturing device 10. See FIG. 4.

It must be observed that, once suturing elements 50, 51 are rigidly joined, there is no possibility of detachment or loosening of the suture.

Thus, the endoscopic suturing device 10 of the present invention is distinguished from the state of the art by providing a pressure joining mechanism such that the approximation under pressure, as well as the rigid association of the suture, simplifies the execution of the surgical procedure, reducing the chances of occurrence of intragastric and extragastric lesions and, consequently, the morbidity rates among patients, as well as reducing the costs of this procedure.

In one embodiment of the invention, the suturing elements 50, 51 comprise a first suturing element 50 rigidly fixed on proximal end 41 of elongated tensionable suturing element 40 and a second suturing element 51 with free movement around and longitudinally to elongated tensionable suturing element 40.

First suturing element 50 presents greater thickness than elongated tensionable suturing element 40, however, smaller than second suturing element 51.

Second suturing element 51 comprises a tubular body shape longitudinal to the elongated tensionable suturing element 40, having an open front part and a rear part with a smaller opening than the front opening, so that second suturing element 51 allows the free movement of the elongated tensionable suturing element 40 inside the second suturing element, but prevents the movement of the first suturing element 50.

Second suturing element 51 also comprises a rear part with an end which corresponds to the limiting region 22 of receptacle 21 so that the set of first suturing element 50 and second suturing element 51 does not move beyond and into elongated tubular element 20.

Receptacle 21 can comprise a limiting region 21 such as a stopper which extends transversely and toward the elongated tensionable suturing element 40, so as to define a passage for the movement of elongated tensionable suturing element 40 while at the same time restricting the movement of suturing elements 50, 51.

Receptacle 21 can comprise an upper opening 23 longitudinal to elongated tensionable suturing element 40, so as to configure the movement of at least one suture thread ST.

Thus, when tensioning element 30 is activated, the rigid union between suturing elements 50, 51 can be configured by the detachment of a first suturing element 50 rigidly fixed on proximal end 41 by cold mechanical interference fit with second suturing element 51 with longitudinal movement limited by limiting region 21. It must be observed that by mechanical interference in the context of the invention, it is understood a mechanical assembly of delicate parts wherein a mechanical pressure is not applied. The main advantages of assembling by interference fit are the reduction in tensions and residual deformities and ability to control the quality of the union.

That is, when tensioning element 30 is activated, the force applied on tension point 31 which is transferred to distal end 42 and to proximal end 41 of elongated tensionable suturing element 40 is greater than the resistance between suturing elements 50, 51 and the elongated tensionable suturing element 40, so that there occurs the detachment of suturing elements 50, 51 from elongated tensionable suturing element 40 together with the assembly by interference fit of suturing elements 50, 51, performing the rigid union between suturing elements 50, 51 and, consequently, the suturing of suture thread ST.

Advantageously, in this embodiment, the first suturing element 50 can further comprise a lower part provided with a surrounding groove so that the force transferred by proximal end 41, which suffices to detach said first suturing element 50 from elongated tensionable suturing element 40, is also sufficient to deform the projection of first suturing element 50 whereby the latter contacts second suturing element 51 and suture thread ST in a shear and cutting configuration of said suture thread ST.

In one embodiment of the present invention, tensioning element 30 comprises a tubular body provided with a longitudinal tensioning internal component associated with an activation point and a longitudinal upper external opening 32. Both the internal tensioning component as the external opening 32 are longitudinal to elongated tensionable suturing element 40.

It must be noted that any embodiments for the longitudinal internal tensioning component associated to an activation point are admissible to the object of the invention, providing the movement to tension the tensionable suturing element is reproduced. A non-exhaustive example corresponds to a rack movement wherein a serrated bar, which uses a gear of the same step, transforms the rotary motion into linear motion.

In this embodiment, the tension point 31 is rigidly fixed on the longitudinal internal tensioning component for fitting with the distal end 41 of elongated tensionable suturing element 40.

In one embodiment of the invention, tension point 31 of tensioning element 30 projects transversely to elongated tensionable suturing element 40, in a split body having lower part 33 rigidly fixed to tensioning element 30 and the upper part 34 operationally able to fit into lower part 33, so as to maintain a rigid coupling between elongated tensionable suturing element 40 and tension point 31.

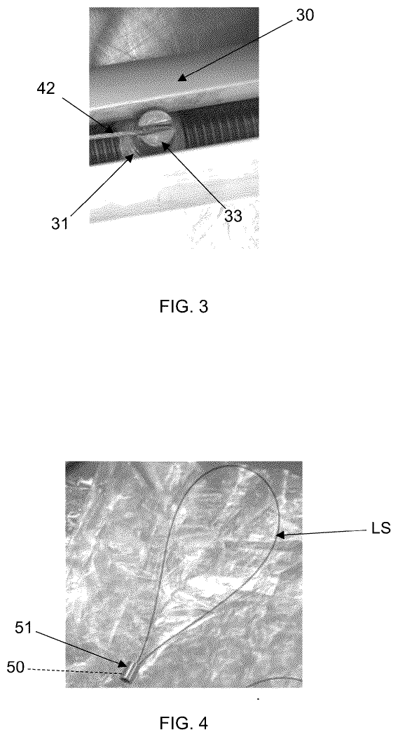

As observed in FIG. 3, tension point 31 can comprise a split body with lower part 33 provided with a slot for fitting with distal end 42 of elongated tensionable suturing element 40.

Thus, advantageously, a health professional can in an easy and practical way perform the movement and tensioning of the elongated tensionable suturing element 40 for rigid and permanent union of suturing elements 50, 51 through the simple activation of the activation point such as a gear rotation movement.

It must be observed that the elements and means inherent to the endoscopic suturing device 10 can be formed from any suitable material, such as stainless steel, titanium, nitinol, or other metals/alloys.

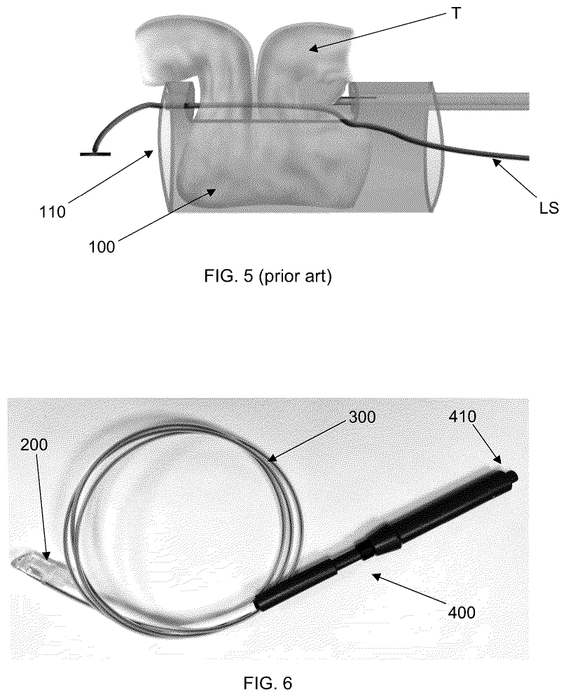

FIG. 5 illustrates an embodiment of the application of the endoscopic suturing device 10 of the present invention together with a suction and suturing chamber 100 of the state of the art.

As observed, the endoscopic suturing device is applicable to a suction and suturing chamber 100 formed by a tubular body which defines an annular side wall, a transversal closing wall 110, the annular side wall defining an internal space comprising an upper opening giving access to the internal space.

The upper opening configuring an area through which the tissue T to be sutured enters the internal space of the body of the suction and suturing chamber 110. The parts of tissue T to be sutured are accommodated under suction created inside the chamber. This suction phenomenon can also be known as vacuum or negative pressure. Reference is made to document BR102021003885-3.

In this context, and in an advantageous manner to that described in document BR102021003885-3, the inventors identified improvements regarding the closing wall of the suturing chamber.

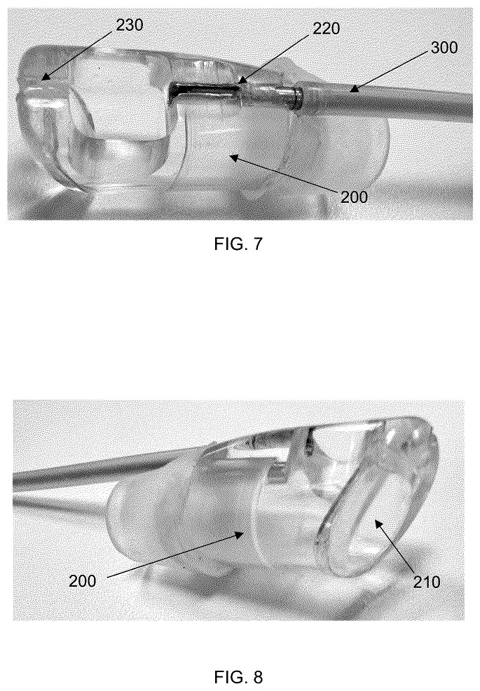

As observed in FIGS. 6, 7 and 8, a configuration of the inclined closing wall 210 in the endoscopic suction and suturing chamber 200 results in less resistance by the suction and suturing chamber 200 during the suction and suturing procedure, apart from an improvement in the suction, visualization, and execution of the suturing during the suction and suturing procedure.

Thus, the present invention also provides an endoscopic suction and suturing chamber 200 comprising an inclined closing wall 210 with a lower part extending between 20° and 80° of the lower end of the annular wall of the suction and suturing chamber 200 to an upper portion of the inclined closing wall 210 and meeting with the upper end of the annular wall of the suction and suturing chamber 200.

Once tissues T are vacuumed, there is inserted a stem 300, by means of an endoscope, in external longitudinal channel 220 of the suction and suturing chamber 200, stem 300 being inserted together with a clip usually known as “T-tag” on the tip of suture thread ST. As reference, said clip is shown in FIG. 5.

Stem 300 together with the clip are conducted along external longitudinal channel 220 until the opening of the suction and suturing chamber 200, wherein they meet the suctioned gastric wall T, which cross the opening of the chamber, passing through the tissues T and enter the longitudinal orifice 230 of suction and suturing chamber 200.

The traversing action is carried out by the release mechanism 400 associated with the other end of stem 300. Advantageously, release mechanism 400 comprises an anatomic shape for use by a health professional in an easy and practical manner, as well as a release button 410 with action to project the clip between the suctioned gastric walls T in a precise and safe manner.

As observed in FIG. 6, the anatomic shape of the release mechanism 400 comprises a tubular body with surface height and area corresponding to the contour of the closing of the hand of the health professional, as well as a projection on the lower portion of the tubular body opposite the release button 410 to provide a support for the base of the hand in a closed configuration. Thus, advantageously, the handling of the release mechanism 400 and the activation by means of the release button 410 can occur with just one hand, with support in a closed configuration together with the free movement of the thumb.

Thus, a junction point is made of the intragastric tissues T, whereby on one side of the tissues there is found the “T-tag” and on the other side the suture thread ST.

The suction and suturing chamber 200 can be once again associated with the stem 300 with a second clip in the shape of a “T-tag” on the tip of a second suture thread ST, executing, in this manner, a second junction point of tissues T.

It must be noted that the chamber 200, as well as stem 300 can be formed of any suitable material such as plastic and translucent materials, such as polycarbonates, polyamides including nylon, polytetrafluoroethylenes (that is, PTFE and EPTFE), polyethylene ether ketones (PEEK), polyvinyl chlorides, polyimides, polyurethanes and polyethylenes. The sutures can be made with common suture material such as known in the art, for example polyester suture such as 4-0 Tevdek®, nylon, silk, polypropylene, ultra-high-molecular-weight polyethylene (UHMPE) and similar. The release mechanism 400 can be formed from any suitable material, such as stainless steel, titanium, nitinol, or other metals/alloys.

Once the points are duly defined, the endoscopic suturing device 10 of the present invention, by means of an endoscope, configures the suturing of suture threads ST.

In this embodiment, the association of the endoscopic suturing device 10 of the present invention with the use of a suction and suturing chamber 200 advantageously results in an improved suturing procedure, wherein the execution time of the procedure is significantly reduced, as well as enabling the union under pressure, so that the pressure approximation makes the sutured tissues merge after a short while.

In one embodiment of the invention, the suturing occurs by means of the insertion of the suture threads ST between suturing elements 50, 51 and the limiting region 22 of receptacle 22 until the longitudinal upper opening 23 of receptacle 22. Thus, the suture threads ST can be freely moved, by means of the upper opening 23, to a tensioned state, for example, by the health professional who performs the suturing procedure.

It must be observed that the execution by the health professional of the tensioning of suture threads ST advantageously enables an improved suture as regards the sensitivity of the professional in identifying and applying due force for joining the stitches of the intragastric T tissues.

Once the suture threads ST are tensioned, the tensioning element 30 of the endoscopic suturing device 10 can be activated so as to “stretch” the distal end 42 of the elongated tensionable suturing element 40 fixed on tension point 31 and causing the suturing elements 50, 51 on the proximal end 41 of the elongated tensionable suturing element 40 to approach the limiting region 22 of receptacle 21 together with suture threads ST.

As previously described, when the length of the elongated tensionable suturing element 40 corresponds to the length of the elongated tubular element 20 and to the distance from the position of the tension point 31 on tensioning element 30, the elongated tensionable suturing element 40 is in a tensioned state.

At this point, suturing elements 50, 51 are superimposed on each other and in a packaging configuration with suture threads ST tensioned, so this set has restricted movement due to limiting region 22 of receptacle 21 and the walls of the intragastric T tissues.

While maintaining the activation of tensioning element 30 of the endoscopic suturing device 10 and in the impossibility of movement of suturing elements 50, 51, the force applied on the tension point 31 which is transferred to the distal end 42 and to the proximal end 41 of the elongated tensionable suturing element 40 is greater than the resistance between the suturing elements 50, 51 and the elongated tensionable suturing element 40, so there occurs the detachment of suturing elements 50, 51 from elongated tensionable suturing element 40, performing the rigid union between suturing elements 50, 51 and, consequently, the suturing between the suture threads ST.

Advantageously, in this embodiment, the first suturing element 50 can further comprise a lower portion provided with a surrounding projection so the force transferred by proximal end 41, which suffices to detach said first suturing element 50 from elongated tensionable suturing element 40, also suffices to deform the projection of first suturing element 50, whereby the latter contacts second suturing element 51 and the suture threads ST in a shear and cutting configuration of said suture threads ST.

Thus, in addition to the rigid union between suturing elements 50, 51 and, consequently, the suturing between the suture threads ST, the endoscopic suturing device 10 of the present invention can cut the suturing threads.

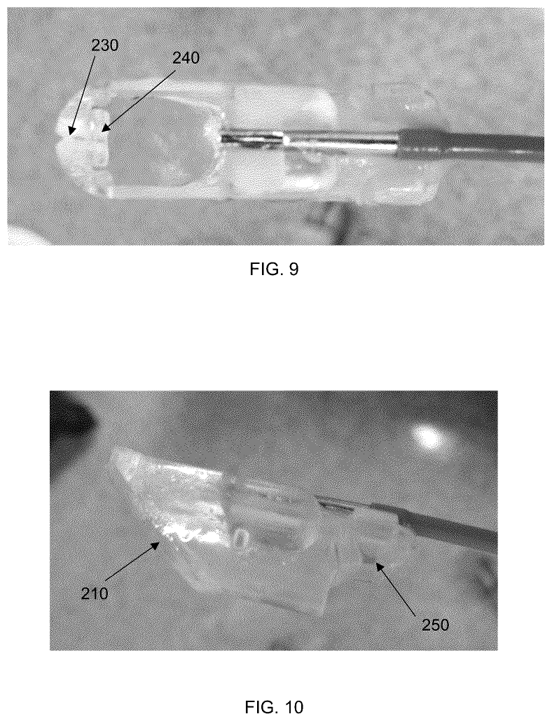

Notwithstanding, as shown in FIG. 9, in one embodiment of the present invention, the endoscopic suction and suturing chamber 200 comprises, on the upper end of the wall, adjacent the longitudinal orifice 230, a projection 240 to accommodate a wire which will be associated to the suture thread ST.

In this embodiment, the wire end is accommodated around the projection 240, in the shape of a bow, so that the clip and the suture thread ST, when crossing the longitudinal orifice 230, also cross the bow formed by the wire end.

Thus, the first and second junction points of T tissues are each provided with a bow on the side opposite suture thread ST, wherein said bows fixed to suture thread ST can be approached and tensioned, for example, manually by the professional, so that the suturing is executed by other means than the endoscopic suturing device 10.

Thus, the configuration of a projection 240 on the wall of the endoscopic suction and suturing chamber 200, adjacent the longitudinal orifice 230, enables the suture to be performed by means of a clipper, in addition to the endoscopic suturing device 10.

After performing the suturing, the remaining wires as well as suture threads ST are removed after being cut with scissors.

Additionally, as shown in FIG. 10, the endoscopic suction and suturing chamber 200 comprises a fit configuration 250 on the part that is opposite the inclined closing wall 210.

In this embodiment, advantageously it is possible to use fastening means, of the elastic type, for the association of the chamber with the endoscope, providing a more robust fastening due to the tensioning of the elastic means, while at the same time allowing ease in the coupling and uncoupling process of the chamber in the endoscope.

It must be observed that, in the context of the present invention, both the first object of the invention and the second object of the invention are associable to an endoscope for obtaining an endoscopic suturing equipment. In other words, the association of the suction and suturing chamber 200 with an endoscope reflects an endoscopic suturing equipment, while at the same time the association of the endoscopic suturing device 10 with an endoscope reflects an endoscopic suturing equipment.

Despite the above description of the preferred embodiments, the present invention can be executed in similar ways, whereby it may present modifications in the manner of implementation, so the scope of protection of the invention is limited only by the contents of the attached claims, including all possible equivalent variations linked to the figures.

1. Endoscopic suturing device (10) comprising an elongated tubular element (20) which extends from a tensioning element (30) associable with an elongated tensionable suturing element (40), wherein the elongated tubular element (20) comprises an inlet end provided with a receptacle (21) with a limiting region (22), the tensioning element (30) comprising a tension point (31) and the elongated tensionable suturing element (40) comprising a proximal end (41) and a distal end (42), wherein the proximal end (41) comprises suturing elements (50, 51), so that the distal end (42) enters the receptacle (21) for fastening to the tension point (31) and the suturing elements (50, 51) are delimited by a limiting region (22) and rigidly joined to each other when the tensioning element (30) is activated.

2. Endoscopic suturing device (10), according to claim 1, wherein the suturing elements (50, 51) configuring a passage for at least one suture thread.

3. Endoscopic suturing device (10), according to claim 1, wherein the suturing elements (50, 51), comprising a first suturing element (50) rigidly fixed on the proximal end (41) and a second suturing element (51) with free movement around and longitudinally to the elongated tensionable suturing element (40).

4. Endoscopic suturing device (10), according to claim 1, wherein the rigid union between the suturing elements (50, 51) being configured by the detachment of a first suturing element (50), rigidly fixed on the proximal end (41) for mechanical interference fit to a second suturing element (51) with longitudinal movement limited by the limiting region (21) when the tensioning element (30) is activated.

5. Endoscopic suturing device (10), according to claim 1, wherein a rigid union between the suturing elements (50, 51) being configured by detachment of a first suturing element (50) comprising a lower portion provided with a surrounding projection.

6. Endoscopic suturing device (10), according to claim 1, wherein the limiting region (21) comprising a stopper which extends transversely and toward the elongated tensionable suturing element (40) so as to define a passage for movement of the elongated tensionable suturing element (40) together with the limitation of movement of the suturing elements (50, 51).

7. Endoscopic suturing device (10), according to claim 1, wherein the receptacle (21) comprising an upper opening (23) longitudinal to the elongated tensionable suturing element (40) so as to configure the movement of at least one suture thread (ST).

8. Endoscopic suturing device (10), according to claim 1, wherein the tensioning element (30) comprising an upper opening (32) longitudinal to the elongated tensionable suturing element (40) for fastening the distal end (42) to the tension point (31).

9. Endoscopic suturing device (10), according to claim 1, wherein the tension point comprising a split body with the lower part (33) rigidly fixed on the tensioning element (30) and the upper part (34) being operationally able to fit into the lower part (33).

10. Endoscopic suturing device (10), according to claim 1, wherein the tension point (31) comprising a split body with the lower part (33) comprising a slot for fastening with the distal end (42).

11. Endoscopic suction and suturing chamber (200), characterized by comprising a tubular body defining an annular side wall and a closing wall, the annular side wall defining an internal space comprising an upper opening which gives access to the internal space, wherein the closing wall is an inclined closing wall (210) with lower portion which extends in an inclined manner as from the lower end of the annular side wall to the upper portion and meeting the upper end of the annular side wall.

12. Endoscopic suction and suturing chamber (200), according to claim 11, characterized by the lower portion of the inclined closing wall (210) extending between 20° and 80° from the lower end of the annular side wall to an upper portion of the inclined closing wall (210).
